(19) United States
(12) Patent Application Publication
(10) Pub. No.: US 2024/0108849 A1
(43) Pub. Date: Apr. 4, 2024

(54) Ultrasonic Catheter System With Longitudinal Pressure Sensing

(71) Applicant: Bard Peripheral Vascular, Inc., Franklin Lakes, NJ (US)

(72) Inventor: Aseem Singh, Tempe, AZ (US)

(73) Assignee: Bard Peripheral Vascular, Inc., Franklin Lakes, NJ (US)

(21) Appl. No.: 18/262,635

(22) PCT Filed: Jan. 29, 2021
(86) PCT No.: PCT/US2021/015816

§ 371 Date: Jul. 24, 2023

(51) Int. Cl. A61M 25/00; A61B 17/22; A61M 25/09

(52) U.S. Cl. CPC A61M 25/00; A61B 17/22; A61M 25/09; A61B 2017/22014; A61M 2025/0002; A61M 2025/09058

(57) ABSTRACT

An ultrasonic catheter system includes an ultrasonic transducer configured to deliver ultrasonic vibrational energy when activated. An ultrasonic catheter is coupled to the ultrasonic transducer to receive the ultrasonic vibrational energy. The ultrasonic catheter includes an elongate flexible catheter sheath and a distal end portion having a longitudinal axis. The distal end portion includes a longitudinal pressure sensor and a distal tip element. The longitudinal pressure sensor is configured to generate a catheter longitudinal pressure signal corresponding to a sensed longitudinal pressure encountered by the ultrasonic catheter along the longitudinal axis of the distal end portion.

### CROSS-REFERENCE TO RELATED APPLICATIONS

None.

### TECHNICAL FIELD

The present invention relates to ultrasonic catheter systems, and more particularly, to an ultrasonic catheter system with sensing of a longitudinal pressure encountered by an ultrasonic catheter.

### BACKGROUND ART

Atherosclerosis is characterized by one or more intravascular occlusions (e.g., lesions) formed in part of plaque including blood-borne substances such as fat, cholesterol, and calcium. An intravascular lesion such as an arterial lesion can form on a side of an arterial lumen and build out across the lumen to an opposite side thereof.

Vascular procedures, such as a crossing procedure or an atherectomy procedure, may be used to restore patency and blood flow that was lost due to one or more intravascular occlusions. A crossing procedure is a procedure in which an opening is formed through the intravascular occlusion. An atherectomy procedure may include crossing, but also attempts to break up and remove the intravascular occlusion. An ultrasonic system having an ultrasonic catheter may be used in performing crossing and atherectomy procedures. For example, vascular occlusion crossing and/or atherectomy, may utilize ultrasonic energy to cross and/or break up the vascular occlusion through the vibration of a catheter tip engaged with the vascular occlusion.

The intravascular occlusion may be, for example, in the form of a calcified vascular occlusion having hard proximal and distal end caps. During an ultrasonic crossing or atherectomy procedure, a distal tip of the ultrasonic catheter engages the proximal end cap of the calcified vascular occlusion. However, because of the hardness of the proximal end cap of the calcified vascular occlusion, the distal tip of the ultrasonic catheter may unintentionally bounce off of the proximal end cap and tend to take a sub-intimal migration path toward or into the side wall of the vasculature.

What is needed in the art is an ultrasonic catheter system with sensing of a longitudinal pressure encountered by an ultrasonic catheter, which may be used to aid in anchoring the distal tip element of the ultrasonic catheter with the proximal end cap of the calcified vascular occlusion following initial engagement of the distal tip element of the ultrasonic catheter with the proximal end cap of the calcified vascular occlusion.

### SUMMARY OF INVENTION

The present invention provides an ultrasonic catheter system with a longitudinal pressure sensor to sense a longitudinal pressure encountered by an ultrasonic catheter, wherein the sensed longitudinal pressure may be used to aid in anchoring the distal tip element of the ultrasonic catheter with the proximal end cap of the calcified vascular occlusion following initial engagement of the distal tip element of the ultrasonic catheter with the proximal end cap of the calcified vascular occlusion.

The invention, in one form, is directed to an ultrasonic catheter system that includes an ultrasonic transducer configured to deliver ultrasonic vibrational energy when activated. An ultrasonic catheter is coupled to the ultrasonic transducer to receive the ultrasonic vibrational energy. The ultrasonic catheter includes an elongate flexible catheter sheath and a distal end portion having a longitudinal axis. The distal end portion includes a longitudinal pressure sensor and a distal tip element. The longitudinal pressure sensor is configured to generate a catheter longitudinal pressure signal corresponding to a sensed longitudinal pressure encountered by the ultrasonic catheter along the longitudinal axis of the distal end portion.

The invention, in another form, is directed to an ultrasonic catheter that includes an elongate flexible catheter sheath and a distal end portion comprising a longitudinal pressure sensor and a distal tip element. The elongate flexible catheter sheath is proximal to the distal tip element. The longitudinal pressure sensor is configured to longitudinally extend between the elongate flexible catheter sheath and the distal tip element.

An advantage of the present invention is that by sensing the longitudinal pressure encountered by, e.g., acting upon, the ultrasonic catheter, an activation of the ultrasonic catheter may be delayed until confirmation of engagement of the distal tip element of the ultrasonic catheter with the occlusion in the blood vessel via the longitudinal pressure sensing, which helps reduce the possibility of subintimal migration of the ultrasonic catheter, and perforation risks, resulting in a greater clinical success rate, and increased safety of the device.

Another advantage of the present invention is that the ultrasonic system may be operated, in at least some applications, so as to selectively activate the ultrasonic transducer only when the ultrasonic catheter encounters a known threshold of resistance with the occlusion, e.g., plaque and/or thrombotic lesion, thereby facilitating precise engagement and application of ultrasonic energy with the hard calcification cap or dense thrombotic tissue by continually sensing the longitudinal pressure encountered by, e.g., acting upon, the ultrasonic catheter during occlusion engagement and crossing.

### BRIEF DESCRIPTION OF DRAWINGS

The above-mentioned and other features and advantages of this invention, and the manner of attaining them, will become more apparent and the invention will be better understood by reference to the following description of an embodiment of the invention taken in conjunction with the accompanying drawings, wherein:

FIG. 1 is a diagrammatic illustration of an ultrasonic catheter system of the present invention;

FIG. 2 is an electrical block diagram of the ultrasonic catheter system of FIG. 1;

FIG. 3 is a side view of a distal end portion of an ultrasonic catheter of the ultrasonic catheter system of FIGS. 1 and 2, wherein the distal end portion includes a distal portion of a catheter sheath, a distal tip element, and a longitudinal pres sure sensor that extends between the catheter sheath and the distal tip element; and

FIG. 4 is a perspective view of the distal end portion of the ultrasonic catheter of FIG. 3.

Corresponding reference characters indicate corresponding parts throughout the several views. The exemplifications set out herein illustrate at least one embodiment of the invention, and such exemplifications are not to be construed as limiting the scope of the invention in any manner.

### DESCRIPTION OF EMBODIMENTS

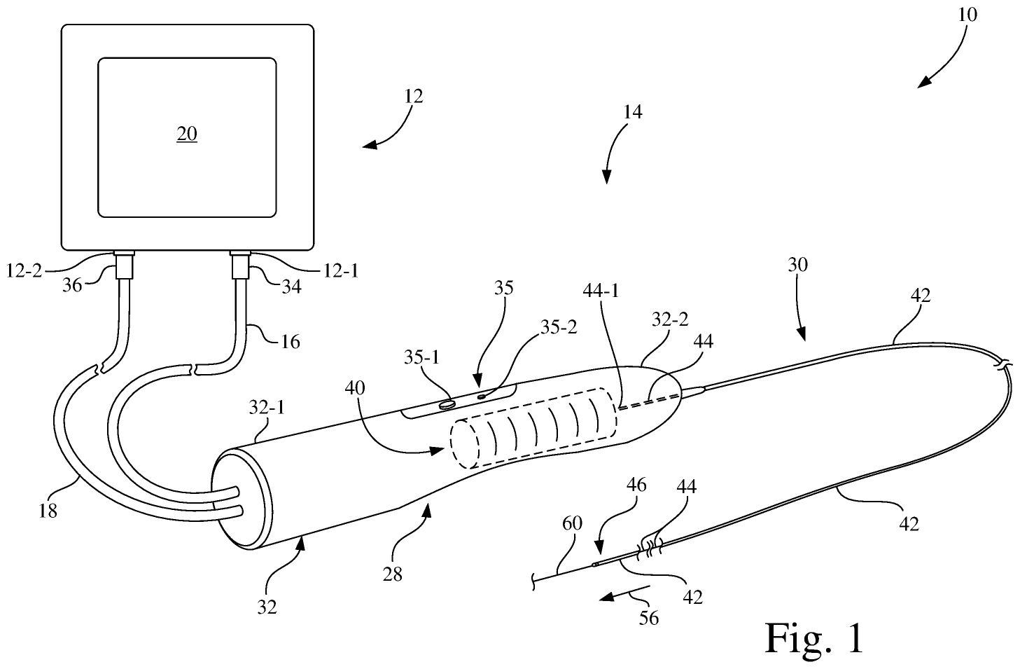

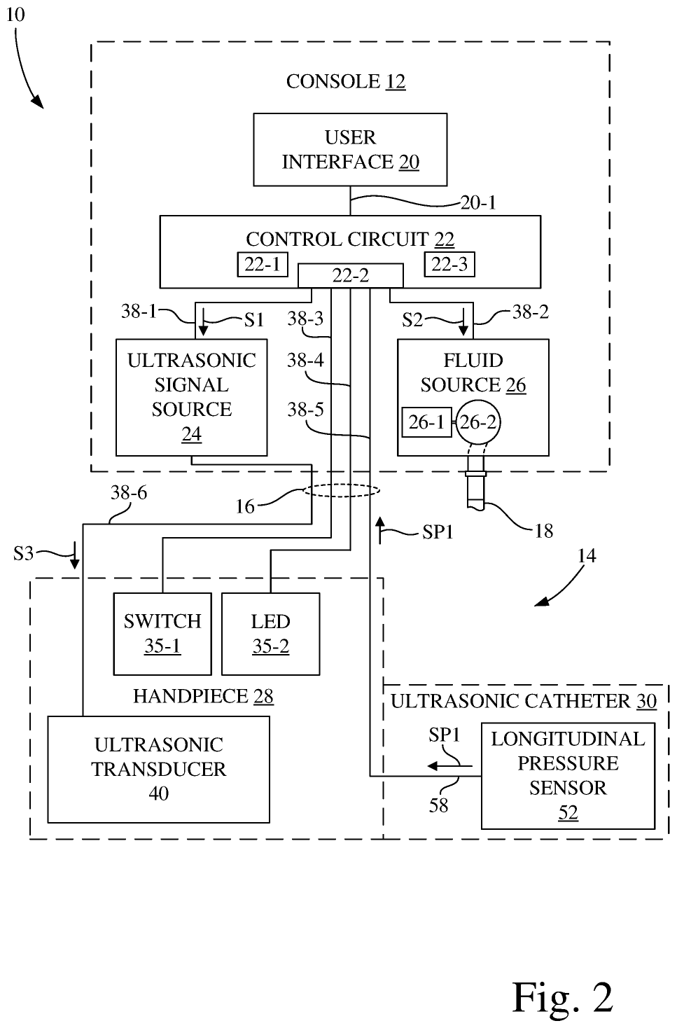

Referring now to the drawings, and more particularly to FIGS. 1 and 2, there is shown an ultrasonic catheter system 10 in accordance with an embodiment of the present invention. Ultrasonic catheter system 10 includes a console 12 and an ultrasonic catheter device 14. Ultrasonic catheter device 14 may be used, for example, for interventional vascular occlusion or oncology procedures.

Console 12 is connected in electrical communication with ultrasonic catheter device 14 via an electrical cable 16, e.g., a multi-conductor cable. Console 12 is connected in fluid communication with ultrasonic catheter device 14 via a fluid conduit 18, e.g., a flexible tube or hose. Console 12 may include multiple components in a single housing unit or in separate housing units.

Referring to FIG. 2, in the present embodiment, console 12 may include a user interface 20, a control circuit 22, an ultrasonic signal source 24, and a fluid source 26. Alternatively, all or a portion of user interface 20, control circuit 22, ultrasonic signal source 24, and fluid source 26 may be located in ultrasonic catheter device 14.

Referring again to FIG. 1, in the present embodiment, ultrasonic catheter device 14 includes a handpiece 28 and an ultrasonic catheter 30, i.e., an ultrasonic vibrational catheter. In the present embodiment, handpiece 28 is permanently connected to ultrasonic catheter 30. Alternatively, however, those skilled in the art will recognize that ultrasonic catheter 30 may be removably connected to ultrasonic catheter 30.

Handpiece 28 includes a housing 32 that has an exterior shape that facilitates being gripped by a user's hand. In the present embodiment, housing 32 has a proximal end 32-1 from which electrical cable 16 and fluid conduit 18 proximally extend. Electrical cable 16 has an electrical connector 34 for connection to a corresponding electrical connector 12-1 of console 12. Fluid conduit 18 has a fluid connector 36 for connection to a corresponding fluid connector 12-2 of console 12. Housing 32 has a distal end 32-2 from which ultrasonic catheter 30 distally extends.

Referring also to FIG. 2, user interface 20 is connected to control circuit 22 via a communication link 20-1, e.g., a multi-wire cable or USB, to provide electrical and communication interconnection. Alternatively, user interface 20 may be a wireless link, e.g., Bluetooth, which is communicatively coupled to control circuit 22. User interface 20 may include, for example, a touchscreen display and associated input and output processing circuitry. Touchscreen display may include, for example, a liquid crystal display (LCD) or a light-emitting diode (LED) display. Alternatively, user interface 20 may be in the form of a laptop computer or tablet. User interface 20 is configured to generate control signals based on user input. For example, a user may operate user interface 20 to provide the control signals to control circuit 22 to initiate and/or terminate operation of ultrasonic signal source 24, and/or to selectively start, stop, or control the fluid feed rate of fluid source 26.

Referring again to FIG. 1, an auxiliary user interface 35 may be mounted to housing 32 of handpiece 28 to provide local control and user feedback. Auxiliary user interface 35 may include an activation switch 35-1 and activation indicator LED 35-2. Activation switch 35-1 is configured to allow the user to activate ultrasonic signal source 24 locally at handpiece 28. For example, activation switch 35-1 may be electrically and communicatively coupled to control circuit 22 via electrical cable 16. Activation indicator LED 35-2 may be configured to provide a visual indication to the user of when handpiece 28 has been activated to generate ultrasonic vibrational energy. Activation indicator LED 35-2 is electrically and communicatively coupled to control circuit 22 via electrical cable 16.

Referring again to FIG. 2, control circuit 22 includes a processor circuit 22-1, interface circuitry 22-2, and an electronic memory circuit 22-3. Control circuit 22 is electrically connected and communicatively coupled to user interface 20 via interface circuitry 22-2 and communication link 20-1, e.g., a multi-wire cable and/or USB. Control circuit 22 is electrically connected and communicatively coupled to ultrasonic signal source 24 via interface circuitry 22-2 and a communication link 38-1, e.g., a multi-wire cable or USB. Control circuit 22 is electrically connected and communicatively coupled to fluid source 26 via interface circuitry 22-2 and a communication link 38-2, e.g., a multi-wire cable or USB. Control circuit 22 is electrically connected and communicatively coupled to activation switch 35-1 via interface circuitry 22-2 and a communication link 38-3, e.g., a multi-wire cable or USB. Control circuit 22 is electrically connected and communicatively coupled to activation indicator LED 35-2 via interface circuitry 22-2 and a communication link 38-4, e.g., a multi-wire cable or USB. Control circuit 22 is electrically connected and communicatively coupled to a longitudinal pressure sensor 52 via interface circuitry 22-2 and a longitudinal pressure communication link 38-5, e.g., a multi-wire cable or USB.

Accordingly, control circuit 22 is electrically connected and communicatively coupled to ultrasonic handpiece 28 of ultrasonic catheter device 14 via interface circuitry 22-2 and a plurality of electrical communication links including a switch communication link 38-3, an LED communication link 38-4, and a longitudinal pressure communication link 38-5, each of which may continue through and/or be incorporated into electrical cable 16. Also included in electrical cable 16 may be a chassis ground (not shown).

Each of the communication links 38-1, 38-2, 38-3, 38-4, and 38-5 may include one or more wire conductors, and is configured to carry respective control signals and/or feedback signals.

Control circuit 22 executes program instructions to process signals, e.g., commands received from user interface 20 and/or to provide display information to user interface 20. Control circuit 22 executes program instructions to provide an output control signal S1 via interface circuitry 22-2 and communication link 38-1 to ultrasonic signal source 24 to control the operation of ultrasonic signal source 24. Control circuit 22 executes program instructions to provide an output control signal S2 via interface circuitry 22-2 and communication link 38-2 to fluid source 26 to control the operation of fluid source 26. Also, control circuit 22 executes program instructions to receive and process a catheter longitudinal pressure signal SP1 received via interface circuit 22-2 and longitudinal pressure communication link 38-5 from longitudinal pressure sensor 52 of ultrasonic catheter 30 of ultrasonic catheter device 14.

Processor circuit 22-1 of control circuit 22 may include one or more programmable microprocessors and associated circuitry, such as an input/output interface, clock, buffers, memory, etc. Processor circuit 22-1 may be programmed, e.g., through software or firmware stored in electronic memory circuit 22-3, to execute program instructions to process received input data from user interface 20 and/or ultrasonic catheter device 14, and to generate and send output data to ultrasonic signal source 24 and/or fluid source 26.

Interface circuitry 22-2 includes input and output circuits to facilitate electrical connection and data transfer with user interface 20, ultrasonic signal source 24, fluid source 26, and ultrasonic catheter device 14.

Electronic memory circuit 22-3 is an electronic non-transitory memory having a plurality of data storage locations, as is well known in the art. Electronic memory circuit 22-3 may be used, for example, to store program instructions to be executed by processor circuit 22-1 of control circuit 22 of console 12, to store longitudinal pressure data associated with catheter longitudinal pressure signal SP1 received from longitudinal pressure sensor 52 of ultrasonic catheter 30 of ultrasonic catheter device 14, and to store a threshold longitudinal resistance pressure value (e.g., as a digital value) corresponding to a threshold longitudinal resistance pressure to which the sensed longitudinal pressure associated with the catheter longitudinal pressure signal SP1 may be compared.

Ultrasonic signal source 24 is typical of that known in the art, and may be controlled via user interface 20 and control circuit 22 to produce, when activated, an ultrasonic electrical signal in the form of an ultrasonic excitation signal S3, e.g., in a frequency range of 20 kHz-40 kHz. Ultrasonic excitation signal S3 is supplied from ultrasonic signal source 24 to handpiece 28 via a communication link 38-6 that continues through and/or is incorporated into electrical cable 16. Communication link 38-6 may include one or more wire conductors, and is configured to carry ultrasonic excitation signal S3.

Fluid source 26 may be, for example, a fluid injector that includes a fluid supply 26-1 and a fluid pump 26-2 that is configured to supply a fluid, such as sterile saline, to ultrasonic catheter 30. The fluid, e.g., sterile saline, may be used, for example, to cool ultrasonic catheter 30 and/or to flush a procedure area in the patient during a medical procedure. Fluid source 26 is connected in fluid communication with handpiece 28 of ultrasonic catheter device 14 via fluid conduit 18.

Handpiece 28 is electrically connected and communicatively coupled to control circuit 22 and to ultrasonic signal source 24 of console 12 via electrical cable 16. In the present embodiment, handpiece 28 contains an ultrasonic transducer 40 that is mounted internally to housing 32. Ultrasonic transducer 40 is connected to ultrasonic signal source 24 via communication link 38-6 of electrical cable 16, wherein ultrasonic transducer 40 converts ultrasonic excitation signal S3 into ultrasonic vibrational energy.

In the present embodiment, ultrasonic transducer 40 may be, for example, a piezoelectric-type transducer. Ultrasonic transducer 40 of handpiece 28 is electrically connected to ultrasonic signal source 24 by electrical cable 16, and more particularly by communication link 38-6 of electrical cable 16, and is configured to receive and convert ultrasonic excitation signal S3 generated by ultrasonic signal source 24 into ultrasonic vibrational energy, which may be in a frequency range corresponding to that of ultrasonic excitation signal S3. For example, if the frequency of ultrasonic excitation signal S3 generated by ultrasonic signal source 24 and supplied to ultrasonic transducer 40 is 20 kHz, then the vibrational frequency of the output of ultrasonic transducer 40 correspondingly may be 20 kHz.

Referring to FIG. 1, ultrasonic catheter 30 is mechanically connected to distal end 32-2 of housing 32 of handpiece 28 of ultrasonic catheter device 14. The connection of ultrasonic catheter 30 to housing 32 of handpiece 28 may be a releasable connection (i.e., temporary), or alternatively, may be a fixed connection (i.e., permanent). In a case wherein ultrasonic catheter device 14 is intended to be fully disposable, then ultrasonic catheter 30 may be permanently attached to handpiece 28, e.g., by overmold, weld, or adhesive. However, if it is desired that handpiece 28 of ultrasonic catheter device 14 be reusable, then ultrasonic catheter 30 may be made to be releasably attachable to handpiece 28, e.g., by a screw coupling or snap connection.

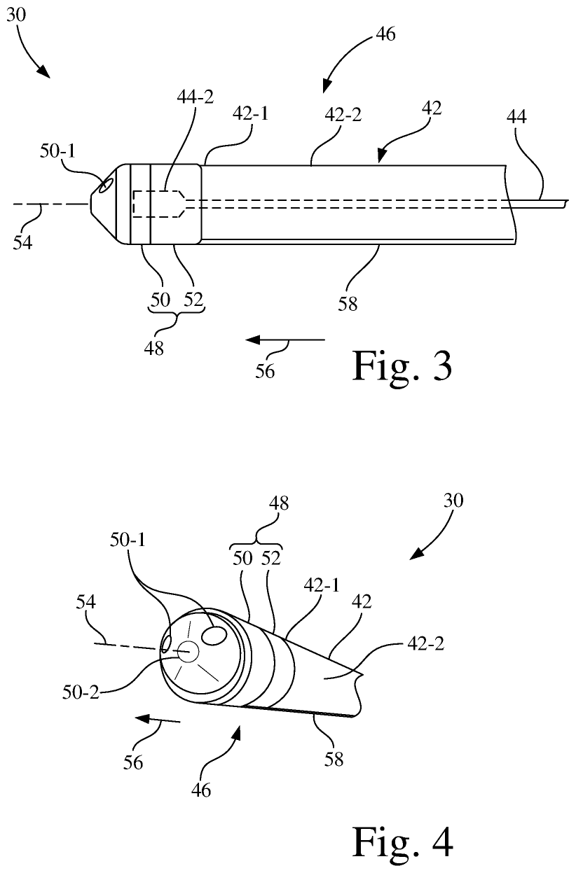

Referring also to FIGS. 2-4, ultrasonic catheter 30 includes an elongate flexible catheter sheath 42, an ultrasonic transmission member 44, and a distal end portion 46. Optionally and alternatively, ultrasonic transmission member 44 may be a component of handpiece 28. Elongate flexible catheter sheath 42 of ultrasonic catheter 30 is mechanically coupled to handpiece 28, e.g., as a releasable connection (i.e., temporary), or alternatively, may be a fixed connection (i.e., permanent). Referring to FIGS. 3 and 4, distal end portion 46 includes a distal body portion 48 that includes a distal tip element 50 and longitudinal pressure sensor 52.

Elongate flexible catheter sheath 42 may be made from a flexible biocompatible material, such as a flexible polymer, which may optionally include structural reinforcement, such as an elongate metal braid or coil. Elongate flexible catheter sheath 42 has a distal end 42-1.

Referring to FIGS. 1-3, ultrasonic transmission member 44 may be in the form of an elongate flexible metal wire, e.g., a nitinol wire, which is sometimes also referred to in the art as a core wire. Ultrasonic transmission member 44 is located in, and longitudinally extends, through elongate flexible catheter sheath 42. Ultrasonic transmission member 44 has a proximal portion 44-1 and a distal portion 44-2 that is longitudinally spaced apart from the proximal portion 44-1.

Proximal portion 44-1 of ultrasonic transmission member 44 is coupled to, and is configured to receive, vibration energy from ultrasonic transducer 40. For example, proximal portion 44-1 of the ultrasonic transmission member 44 may be mechanically connected to ultrasonic transducer 40, e.g., by a sonic connector, to receive the vibrational energy from ultrasonic transducer 40 so as to produce a vibrational motion of ultrasonic transmission member 44. Thus, ultrasonic transducer 40 generates vibratory energy at a vibratory energy level corresponding to the electrical energy output level of the ultrasonic excitation signal S3 generated by ultrasonic signal source 24.

Distal portion 44-2 of ultrasonic transmission member 44 is coupled to, and is configured to deliver, the vibration energy from ultrasonic transducer 40 to distal tip element 50. In the present embodiment, distal tip element 50 is mechanically connected, e.g., soldered, welded, or crimped, to distal portion 44-2 of ultrasonic transmission member 44. Distal tip element 50 is made of a metal, such as stainless steel. Referring also to FIG. 4, in the present embodiment, distal tip element 50 includes one or more fluid lumen(s) 50-1 and a guidewire lumen 50-2. The one or more fluid lumen(s) 50-1 is/are coupled in fluid communication with fluid source 26 via one or more fluid passages in elongate flexible catheter sheath 42 and fluid conduit 18. Distal tip element 50 has a rounded and/or tapered distal surface that is configured, e.g., in size and shape, to engage an occlusion in a blood vessel when ultrasonic catheter 30 is moved in a distal direction 56 into and through the blood vessel.

Longitudinal pressure sensor 52 may be longitudinally joined to distal tip element 50, e.g., by adhesive. Also, longitudinal pressure sensor 52 may be formed along and/or around a flexible substrate, e.g., a tube, of distal body portion 48. In the present embodiment, for example, longitudinal pressure sensor 52 may longitudinally extend (e.g., may be interposed) between elongate flexible catheter sheath 42 and distal tip element 50, and may be respectively connected to distal end 42-1 of elongate flexible catheter sheath 42 and to a proximal surface of distal tip element 50, e.g., by adhesive.

Longitudinal pressure sensor 52 is configured to generate catheter longitudinal pressure signal SP1 that corresponds to a sensed longitudinal pressure encountered by ultrasonic catheter 30 along a longitudinal axis 54 (see FIGS. 3 and 4) of distal end portion 46 of ultrasonic catheter 30. Longitudinal pressure sensor 52 is a pressure transducer that converts pressure into an electrical signal, e.g., analog or digital, represented as catheter longitudinal pressure signal SP1. Such pressure transducers suitable for use as longitudinal pressure sensor 52, may be, for example, a resistive pressure transducer, a capacitive pressure transducer, digital output pressure transducer, voltage/current output pressure transducer, etc. However, common to all of these types of pressure transducers is that the conversion of pressure into an electrical signal is achieved by the physical deformation of a portion of the pressure transducer, which then produces, for example, an electrical resistance, or a change in electrical resistance, that is proportional to the pressure that causes the physical deformation.

In one embodiment, for example, longitudinal pressure sensor 52 may be made out of composite conductive material having a flexible substrate. In the present embodiment, longitudinal pressure sensor 52 is interposed between, and connected to each of, distal tip element 50 and distal end 42-1 of elongate flexible catheter sheath 42. Stated differently, two “anchors” of the flex MEMS sensor circuit of longitudinal pressure sensor 52 is placed relative to distal tip element 50 and distal end 42-1 of elongate flexible catheter sheath 42. Longitudinal pressure sensor 52 may be, for example, in the form of a commercially available strain gauge, wherein an electrical resistance of the strain gauge varies with an amount of applied force (e.g., compression of tension). Thus, in the present embodiment, catheter longitudinal pressure signal SP1 may be a voltage and/or current representation (analog) or a digital representation of the present electrical resistance of longitudinal pressure sensor 52.

Accordingly, such a strain gauge as longitudinal pressure sensor 52 may sense a longitudinal pressure (e.g., a longitudinal (e.g., axial) component of force) applied to distal tip element 50 of ultrasonic catheter 30 based on an amount of deflection (e.g., compression and/or tension) of distal tip element 50 relative to distal end 42-1 of elongate flexible catheter sheath 42. Thus, the amount of deflection of distal tip element 50 relative to the distal end 42-1 of elongate flexible catheter sheath 42 results in a compression and/or tension applied to longitudinal pressure sensor 52, which in turns represents the amount of deflection as a change of resistance of longitudinal pressure sensor 52, and this change in electrical resistance will be indicative of the sensed longitudinal pressure encountered by, e.g., acting upon, ultrasonic catheter 30 along a longitudinal axis 54 (see FIGS. 3 and 4) of distal end portion 46, e.g., encountered by distal tip element 50 of ultrasonic catheter 30.

Thus, the amount of sensed longitudinal pressure encountered by ultrasonic catheter 30 may be used to indicate the presence of and/or engagement with an occlusion in a blood vessel, and may serve as a gauge of the degree of occlusion in a blood vessel. Stated differently, the amount of deflection of distal tip element 50 relative to the distal end 42-1 of elongate flexible catheter sheath 42 may be used to indicate the presence of and/or engagement of distal tip element 50 with an occlusion in a blood vessel. Also, the amount of sensed longitudinal pressure encountered by ultrasonic catheter 30 may serve as a gauge of the degree of occlusion in a blood vessel and/or as a gauge of a force being applied to the occlusion by distal tip element 50 as the user pushes ultrasonic catheter 30 in distal direction 56 during a procedure. The range of deflection may be from slight deflection to large deflection depending on the amount of longitudinal force that the physician is applying to ultrasonic catheter 30 as distal tip element 50 engages the occlusion.

Control circuit 22 may then execute program instructions to process catheter longitudinal pressure signal SP1, e.g., as a representation of the present electrical resistance of longitudinal pressure sensor 52, and control circuit 22 may then compare a value (e.g., a digital value) of the catheter longitudinal pressure signal SP1 with the threshold longitudinal resistance pressure value stored in electronic memory circuit 22-3. In one embodiment, control circuit 22 may be configured, e.g., to execute program instructions to activate ultrasonic transducer 40 to generate ultrasonic vibrational energy when the value of the catheter longitudinal pressure signal SP1, i.e., the sensed longitudinal pressure, from longitudinal pressure sensor 52 of ultrasonic catheter 30 is equal to or greater than the threshold longitudinal resistance pressure value, e.g., a predefined threshold longitudinal resistance pressure. Accordingly, in one application of the present embodiment, the generation of the ultrasonic vibrational energy may begin and/or be sustained only when the sensed longitudinal pressure encountered by, e.g., acting upon, ultrasonic catheter 30, as represented by catheter longitudinal pressure signal SP1, is equal to or greater than the amount of the threshold longitudinal resistance pressure as represented by the threshold longitudinal resistance pressure value stored in electronic memory circuit 22-3.

In another embodiment, longitudinal pressure sensor 52 may be, for example, a micro-electromechanical system (MEMS) piezoelectric transducer array, wherein piezoelectric microrods may be periodically embedded in a passive epoxy matrix. Longitudinal pressure sensor 52 may be integrated into distal end portion 46 of ultrasonic catheter 30 to sense vibration as an indication of a sensed longitudinal pressure (e.g., deflection) at distal end portion 46. A correlation between the sensed vibration and sensed longitudinal pressure (e.g., deflection) at distal end portion 46 may be determined by control circuit 22 with reference to empirical data stored in electronic memory circuit 22-3. The amount of vibration, and thus sensed longitudinal pressure, may be in a range of slight to large depending on the amount of longitudinal force that the physician is applying to ultrasonic catheter 30. The determined amount of sensed longitudinal pressure indicates the presence of an occlusion and/or may serve as a gauge of the degree of occlusion in a blood vessel.

Ultrasonic catheter 30 includes a set of electrical conductors 58 that join and form an extension of longitudinal pressure communication link 38-5 at or within handpiece 28 so as to form a continuous electrical communication link from longitudinal pressure sensor 52 to control circuit 22. The set of electrical conductors 58 extend proximally from longitudinal pressure sensor 52 along a length, e.g., the entire length, of elongate flexible catheter sheath 42 to handpiece 28. In the present embodiment, the set of electrical conductors 58 are attached to an elongate body 42-2 of elongate flexible catheter sheath 42. Alternatively, however, all or a portion of the set of electrical conductors 58 may be embedded within the elongate body 42-2 of elongate flexible catheter sheath 42.

In any of the embodiments, control circuit 22 is electrically coupled to longitudinal pressure sensor 52 and is electrically coupled to ultrasonic transducer 40. In one exemplary implementation, control circuit 22 may be configured, e.g., through execution of program instructions, to activate ultrasonic transducer 40 when a sensed longitudinal pressure from longitudinal pressure sensor 52 of ultrasonic catheter 30, e.g., as represented by a value of catheter longitudinal pressure signal SP1, is equal to or greater than a threshold longitudinal resistance pressure as represented by a threshold longitudinal resistance pressure value. The threshold longitudinal resistance pressure value is predetermined and may be stored as a digital value in electronic memory circuit 22-3 for comparison to the value catheter longitudinal pressure signal SP1, e.g., a digital value, in digital form. Alternatively, those skilled in the art will recognize that the comparison may alternatively be implemented using analog electronic components, if desired.

Thus, with reference also to FIG. 1, according to one application of the present invention, while activation switch 35-1 may be configured to enable the user to activate ultrasonic signal source 24 locally at handpiece 28, the activation of ultrasonic transducer ultrasonic transducer 40 may occur only when a sensed longitudinal pressure from longitudinal pressure sensor 52 of ultrasonic catheter 30, e.g., as represented by a value of the catheter longitudinal pressure signal SP1, is equal to or greater than a threshold longitudinal resistance pressure as represented by the threshold longitudinal resistance pressure value stored in electronic memory circuit 22-3. Thus, ultrasonic catheter system 10 will aid in anchoring distal tip element 50 of ultrasonic catheter 30 with a proximal end cap of a calcified vascular occlusion following initial engagement of distal tip element 50 of ultrasonic catheter 30 with the proximal end cap of the calcified vascular occlusion by delaying and/or sustaining the activation of ultrasonic transducer 40 until when distal tip element 50 of ultrasonic catheter 30 is firmly in contact with the calcified vascular occlusion, i.e., until when the sensed longitudinal pressure from longitudinal pressure sensor 52 of ultrasonic catheter 30, e.g., as represented by a value of the catheter longitudinal pressure signal SP1, is equal to or greater than the threshold longitudinal resistance pressure as represented by the threshold longitudinal resistance pressure value.

Activation indicator LED 35-2 at handpiece 28 may be configured (used) to provide a visual indication to the user of ultrasonic catheter system 10 that ultrasonic transducer ultrasonic transducer 40 has been activated to generate ultrasonic vibrational energy.

In the present embodiment, as depicted in FIG. 1, ultrasonic catheter 30 includes guidewire lumen 50-2 that is configured to facilitate optional use of ultrasonic catheter 30 with a guidewire 60. Guidewire lumen 50-2 is configured, e.g., in size and in shape, to slidably receive guidewire 60. In other words, ultrasonic catheter 30 may be used with guidewire 60 or without guidewire 60, depending upon the procedure and/or preference of the physician using ultrasonic catheter device 14. Guidewire 60 may be, for example, an elongate flexible member that may be, for example, made from a memory material, such as nitinol.

The following items also relate to the invention:

In one embodiment, the invention relates to an ultrasonic catheter system that comprises an ultrasonic transducer and an ultrasonic catheter. The ultrasonic transducer may be configured to deliver ultrasonic vibrational energy when activated. The ultrasonic catheter may be coupled to the ultrasonic transducer to receive the ultrasonic vibrational energy. The ultrasonic catheter may include an elongate flexible catheter sheath and a distal end portion having a longitudinal axis. The distal end portion may include a longitudinal pressure sensor (i.e., a sensor that senses longitudinal pressure) and a distal tip element. The longitudinal pressure sensor may be configured to generate a catheter longitudinal pressure signal that corresponds to a sensed longitudinal pressure encountered by the ultrasonic catheter along the longitudinal axis of the distal end portion.

In the embodiment described in the immediately preceding paragraph, the elongate flexible catheter sheath is proximal to the distal tip element. The longitudinal pressure sensor may be configured to longitudinally extend between the elongate flexible catheter sheath and the distal tip element.

In the embodiment described in any one of the immediately preceding two paragraphs, the ultrasonic catheter system may comprise a control circuit electrically coupled to the longitudinal pressure sensor and to the ultrasonic transducer. The control circuit may be configured to activate the ultrasonic transducer when the sensed longitudinal pressure from the longitudinal pressure sensor of the ultrasonic catheter is equal to or greater than a threshold longitudinal resistance pressure.

In the embodiment described in the immediately preceding paragraph, the control circuit may include an electronic memory that stores a threshold longitudinal resistance pressure value that corresponds to the threshold longitudinal resistance pressure. The control circuit may be configured to execute program instructions to compare a value of the catheter longitudinal pressure signal generated by the longitudinal pressure sensor to the threshold longitudinal resistance pressure value. The control circuit may be configured to execute program instructions to activate the ultrasonic transducer to generate the ultrasonic vibrational energy (only) when the value of the catheter longitudinal pressure signal is equal to or greater than the threshold longitudinal resistance pressure value.

In the embodiment described in any one of the immediately preceding two paragraphs, the ultrasonic catheter system may comprise a set of electrical conductors that may be coupled in electrical communication with each of the longitudinal pressure sensor and the control circuit.

In the embodiment described in the immediately preceding paragraph, the set of electrical conductors may extend proximally from the longitudinal pressure sensor along a length of the elongate flexible catheter sheath.

In accordance with any of the preceding embodiments, the ultrasonic catheter system may comprise a handpiece that contains the ultrasonic transducer. The elongate flexible catheter sheath of the ultrasonic catheter may be coupled to the handpiece.

In accordance with any of the preceding embodiments, the longitudinal pressure sensor may be a pressure transducer configured to convert pressure into an electrical signal by a physical deformation of a portion of the pressure transducer, which then produces the catheter longitudinal pressure signal, wherein the catheter longitudinal pressure signal may be proportional to the pressure that causes the physical deformation.

In accordance with any of the preceding embodiments, the ultrasonic catheter system may comprise an activation indicator LED that may be configured to provide a visual indication to a user of an activation of the ultrasonic transducer.

In accordance with any of the preceding embodiments, the ultrasonic catheter system may comprise a guidewire. The ultrasonic catheter may include a guidewire lumen that may be configured to receive the guidewire.

In another embodiment, the invention relates to an ultrasonic catheter that comprises an elongate flexible catheter sheath and a distal end portion that has a longitudinal axis. The distal end portion may comprise a longitudinal pressure sensor (i.e., a sensor that senses longitudinal pressure) and a distal tip element, wherein the elongate flexible catheter sheath is proximal to the distal tip element. The longitudinal pressure sensor may be configured to longitudinally extend between the elongate flexible catheter sheath and the distal tip element.

In the embodiment described in the immediately preceding paragraph, the longitudinal pressure sensor may be configured to generate a catheter longitudinal pressure signal that corresponds to a sensed longitudinal pressure encountered by the ultrasonic catheter along the longitudinal axis of the distal end portion.

In the embodiment described in any one of the immediately preceding two paragraphs, the ultrasonic catheter may comprise a set of electrical conductors coupled in electrical communication with the longitudinal pressure sensor. The set of electrical conductors may extend proximally from the longitudinal pressure sensor along a length of the elongate flexible catheter sheath.

In the embodiment described in the immediately preceding paragraph, the set of electrical conductors may be attached to (an outer surface of) an elongate body of the elongate flexible catheter sheath.

As one alternative to the embodiment described in the immediately preceding paragraph, the set of electrical conductors may be embedded within (a side wall of) an elongate body of the elongate flexible catheter sheath.

In accordance with any of the preceding embodiments, the longitudinal pressure sensor may be a pressure transducer configured to convert pressure into an electrical signal by a physical deformation of a portion of the pressure transducer. The longitudinal pressure sensor may be configured to produce a catheter longitudinal pressure signal proportional to the pressure that causes the physical deformation.

In accordance with any of the preceding embodiments, the ultrasonic catheter may comprise a guidewire lumen that may be configured to slidably receive a guidewire.

Words of degree are relative modifiers intended to indicate permissible variation from the characteristic so modified. Such terms are not intended to be limited to the absolute value of the characteristic which it modifies, but rather possessing more of the physical or functional characteristic than the opposite, and approaching or approximating such a physical or functional characteristic.

Also, as used herein, the term “coupled”, and its derivatives, is intended to embrace any operationally functional connection, i.e., a direct connection or an indirect connection.

While this invention has been described with respect to at least one embodiment, the present invention can be further modified within the spirit and scope of this disclosure. This application is therefore intended to cover any variations, uses, or adaptations of the invention using its general principles. Further, this application is intended to cover such departures from the present disclosure as come within known or customary practice in the art to which this invention pertains and which fall within the limits of the appended claims.

1. An ultrasonic catheter system, comprising:
an ultrasonic transducer configured to deliver ultrasonic vibrational energy when activated; and
an ultrasonic catheter coupled to the ultrasonic transducer to receive the ultrasonic vibrational energy, the ultrasonic catheter including an elongate flexible catheter sheath and a distal end portion having a longitudinal axis, the distal end portion including a longitudinal pressure sensor and a distal tip element, the longitudinal pressure sensor configured to generate a catheter longitudinal pressure signal corresponding to a sensed longitudinal pressure encountered by the ultrasonic catheter along the longitudinal axis of the distal end portion.

2. The ultrasonic catheter system according to claim 1, wherein the elongate flexible catheter sheath is proximal to the distal tip element, and wherein the longitudinal pressure sensor is configured to longitudinally extend between the elongate flexible catheter sheath and the distal tip element.

3. The ultrasonic catheter system according to claim 1, further comprising a control circuit electrically coupled to the longitudinal pressure sensor and to the ultrasonic transducer, the control circuit configured to activate the ultrasonic transducer when the sensed longitudinal pressure from the longitudinal pressure sensor of the ultrasonic catheter is equal to or greater than a threshold longitudinal resistance pressure.

4. The ultrasonic catheter system according to claim 3, wherein the control circuit includes an electronic memory that stores a threshold longitudinal resistance pressure value corresponding to the threshold longitudinal resistance pressure,
the control circuit is configured to execute program instructions to compare a value of the catheter longitudinal pressure signal generated by the longitudinal pressure sensor to the threshold longitudinal resistance pressure value, and
the control circuit is configured to execute program instructions to activate the ultrasonic transducer to generate the ultrasonic vibrational energy when the value of the catheter longitudinal pressure signal is equal to or greater than the threshold longitudinal resistance pressure value.

5. The ultrasonic catheter system according to claim 3, comprising a set of electrical conductors coupled in electrical communication with each of the longitudinal pressure sensor and the control circuit.

6. The ultrasonic catheter system according to claim 5, wherein the set of electrical conductors extends proximally from the longitudinal pressure sensor along a length of the elongate flexible catheter sheath.

7. The ultrasonic catheter system according to claim 1, comprising a handpiece that contains the ultrasonic transducer, and wherein the elongate flexible catheter sheath of the ultrasonic catheter is coupled to the handpiece.

8. The ultrasonic catheter system according to claim 1, wherein the longitudinal pressure sensor is a pressure transducer configured to convert pressure into an electrical signal by a physical deformation of a portion of the pressure transducer, which then produces the catheter longitudinal pressure signal, wherein the catheter longitudinal pressure signal is proportional to the pressure that causes the physical deformation.

9. The ultrasonic catheter system according to claim 1, comprising an activation indicator LED that is configured to provide a visual indication to a user of an activation of the ultrasonic transducer.

10. The ultrasonic catheter system according to claim 1, comprising a guidewire, and wherein the ultrasonic catheter includes a guidewire lumen that is configured to receive the guidewire.

11. An ultrasonic catheter, comprising:
an elongate flexible catheter sheath; and
a distal end portion having a longitudinal axis, the distal end portion comprising a longitudinal pressure sensor and a distal tip element, wherein the elongate flexible catheter sheath is proximal to the distal tip element, and wherein the longitudinal pressure sensor is configured to longitudinally extend between the elongate flexible catheter sheath and the distal tip element.

12. The ultrasonic catheter according to claim 11, wherein the longitudinal pressure sensor is configured to generate a catheter longitudinal pressure signal corresponding to a sensed longitudinal pressure encountered by the ultrasonic catheter along the longitudinal axis of the distal end portion.

13. The ultrasonic catheter according to claim 11, comprising a set of electrical conductors coupled in electrical communication with the longitudinal pressure sensor, wherein the set of electrical conductors extends proximally from the longitudinal pressure sensor along a length of the elongate flexible catheter sheath.

14. The ultrasonic catheter according to claim 13, wherein the set of electrical conductors is attached to an elongate body of the elongate flexible catheter sheath.

15. The ultrasonic catheter according to claim 13, wherein the set of electrical conductors is embedded within an elongate body of the elongate flexible catheter sheath.

16. The ultrasonic catheter according to claim 11, wherein the longitudinal pressure sensor is a pressure transducer configured to convert pressure into an electrical signal by a physical deformation of a portion of the pressure transducer, the longitudinal pressure sensor configured to produce a catheter longitudinal pressure signal proportional to the pressure that causes the physical deformation.

17. The ultrasonic catheter according to claim 11, comprising a guidewire lumen that is configured to slidably receive a guidewire.
